(19) United States
(12) Patent Application Publication
(10) Pub. No.: US 2001/0029025 A1
(43) Pub. Date: Oct. 11, 2001

(54) Method of identifying proteins

(76) Inventor: Gideon Dreyfuss, Wynnewood, PA (US)

Correspondence Address: MORGAN, LEWIS & BOCKIUS, 1800 M STREET NW, WASHINGTON, DC, 20036-5869, US

(21) Appl. No.: 09/781,773

(22) Filed: Feb. 12, 2001

Related U.S. Application Data
(63) Continuation of application No. PCT/US99/18005, filed on Aug. 13, 1999.
(60) Provisional application No. 60/096,383, filed on Aug. 13, 1998.

(51) Int. Cl. C12Q 1/70; G01N 33/53

(52) U.S. Cl. 435/7.1; 435/5

(57) ABSTRACT

The invention relates to a method of isolating an interacting protein.

### FIELD OF THE INVENTION

The field of the invention is the identification of interacting proteins.

### BACKGROUND OF THE INVENTION

Knowledge of the proteins with which a protein interacts is often the key to understanding its function. Biochemical approaches that rely on protein purification have been extensively used to identify and subsequently clone interacting proteins. Several methods designed to facilitate this process have been developed and used for a wide range of applications in molecular and cell biology, including yeast two-hybrid screens (Fields and Song, 1989, Nature 340:245-246; Chien et al., 1991, Proc. Natl. Acad. Sci. USA, 88:9578-9582; Gyuris et al., 1993, Cell 75:791-803; Durkee et al., 1993, Genes & Development 7:555-569; M. Fromont-Rachine et al., 1997, Nat Genet. 16:277-282), phage display (Smith, 1985, Science 228:1315-1217; Smith, 1991, Curr. Opin. Biotechnol. 2:668-673; Hanes and Pluckthun, 1997, Proc. Natl. Acad. Sci. USA 94:4937-4942) and polysome selection (Schechter, 1973, Proc. Natl. Acad. Sci. USA 70:2256-2260; Payvar and Schimke, 1979, Eur. J. Biochem. 101:271-282; Kraus and Rosenberg, 1982, Proc. Natl. Acad. Sci. USA 79:4015-4019; Mattheakis and Bhatt, 1994, Proc. Natl. Acad. Sci. USA 91:9022-9026; Roberts and Szostak, 1997, Proc. Natl. Acad. Sci. USA 94:12297-12302). In our own work, we have encountered several difficulties with some of these methods. For example, using RNA-binding proteins as baits in yeast two-hybrid screens often resulted in non-specific cloning of other RNA-binding proteins because of RNA bridging. For nuclear transport receptors such as transportin (Pollard et al., 1996, Cell 86:985-994), transport substrates could not be identified by two-hybrid screens. For some members of this receptor family the alternative biochemical route was also difficult as producing them in bacteria was often unsuccessful, and even when successful, required microsequencing of candidate interactors before obtaining clones was possible.

There is a need in the art for a rapid and accurate method of identifying interacting proteins. The present invention satisfies this need.

### SUMMARY OF THE INVENTION

The invention includes a method of isolating an interacting protein. The method comprises contacting a bait protein with a phage display library, wherein the phage display library comprises candidate interacting proteins expressed on the surface of phage in the library, separating phage in the phage display library which bind the bait protein from phage in the phage display library which do not bind the bait protein, and isolating the interacting protein from the phage which bind the bait protein.

In one aspect, the bait protein is obtained by in vitro translation of RNA encoding the bait protein.

In one embodiment, the bait protein has a detectable label attached thereto. More preferably, the detectable label is biotinylated lysine.

In another embodiment, the bait protein is encoded in a gene fused to a gene encoding a binding protein. More preferably, the binding protein is glutathione S transferase.

In another aspect, the phage display library comprises bacteriophage T7.

The invention further includes a method of obtaining an interacting protein capable of binding to a bait protein with high affinity. The method comprises contacting a bait protein with a phage display library, wherein the phage display library comprises candidate interacting proteins expressed on the surface of phage in the library, separating phage in the phage display library which bind the bait protein from phage in the phage display library which do not bind the bait protein, isolating DNA encoding the interacting protein from the phage which bind the bait protein, mutating the DNA to generate an interacting protein encoded thereby which is capable of binding to the bait protein with high affinity.

### BRIEF DESCRIPTION OF THE DRAWINGS

FIG. 1 is a diagram of a novel system for cloning interacting proteins using a biotinylated in vitro translated protein as a bait for selection of interacting proteins expressed on the surface of bacteriophage T7.

FIG. 2 is an image of a gel depicting the size of PCR-amplified cDNA inserts from the T7 phage display HeLa library. DNA was prepared from 18 randomly picked phage plaques, and the inserts were amplified by PCR using T7 specific primers flanking the cloning site. Amplified fragments were resolved by electrophoresis on a 1.2% agarose gel and visualized by staining with ethidium bromide.

FIG. 3 is an image of a blot depicting the translated product, biotin-TRN1. Production of biotin-containing TRN1 by in vitro transcription-translation. Translation was carried out in a coupled transcription-translation rabbit reticulocyte lysate system supplemented with biotin-lysine tRNA, with or without addition of a plasmid containing full-length TRNI. Biotinylated proteins were produced in a coupled in vitro transcription translation system (Promega TNT) containing 25 microliters rabbit reticulocyte lysate, 2 microliters TNT buffer, 1 microliters T7 RNA polymerase, 1 microliter RNasin RNase inhibitor, 1 microliter biotin-RNA-lysine (10 mM)(Boehringher Mannheim #1559478), 1 microliter Amino acid mix without lysine (50 mM)(Boehringher Mannheim #1559478), 1 microliter DNA plasmid (1 μg/μl), and 17 microliters distilled H₂O to a final volume of 50 microliters. Incubation was at 30° C. for 1.5 hours. A three microliter aliquot of the translation mix was resolved by SDS-PAGE and the proteins were transferred to a nitrocellulose membrane by electroblotting. Biotinylated proteins were detected with horseradish peroxidase-conjugated streptavidin and an ECL detection kit.

FIG. 4 is a series of images depicting the results of filter hybridization with A1 and A2 cDNAs on similar number of plated plaques for the original library after consecutive rounds of selection with biotin-translated TRN1. Hybridizations were carried out using full-length cDNAs of hnRNP A1 and A2 (Burd et al., 1989, Proc. Natl. Acad. Sci. USA 86:9788-9792).

FIG. 5 is a table identifying 20 randomly picked phage plaques after four rounds of selection with biotin-TRN1.

FIG. 6 is a table identifying 10 randomly picked phage plaques after six rounds of selection with GST-TRN1.

### DETAILED DESCRIPTION

The invention is based on discovery of a novel assay system for identification of proteins which interact with one another. As noted herein, each of the prior art methods of identifying interacting proteins has limitations which the present method overcomes. In one embodiment, the present method involves the generation of an in vitro translated protein having a detectable tag which protein is referred to herein as a “bait” protein. In a second embodiment, the present method involves a fusion construct wherein, the gene encoding the bait protein is fused to a sequence encoding a binding protein. The method also involves the generation of a phage display library comprising cDNAs obtained from a desired source. Each phage in the phage display library comprises a protein encoded by the cDNA contained therein, which protein is displayed on the surface of the phage. The bait protein is contacted with the phage display library, and proteins expressed on the surface of the phage which interact with the bait protein, bind to the bait protein. Phage having bait protein so bound are separated from phage which do not have protein bound thereon. DNA encoding the phage displayed protein is obtained and is used to generate the protein which interacts with the bait protein.

Interacting proteins identified by the method of the invention are useful for diagnostic and therapeutic purposes.

DNA encoding the interacting protein may then be cloned, sequenced and the DNA and the protein may be subsequently characterized using methodology common in the arts of molecular biology, cell biology, immunology, and the like.

### Definitions

The articles “a” and “an” are used herein to refer to one or more than one (i.e. to at least one) of the grammatical object of the article. By way of example, “an element” means one element or more than one element.

A first nucleic acid region and a second nucleic acid region are arranged in an “antiparallel” fashion if, when the first region is fixed in space and extends in a direction from its 5′-end to its 3′-end, at least a portion of the second region lies parallel to the first strand and extends in the same direction from its 3′-end to its 5′-end.

“Amplification” refers to any means by which a polynucleotide sequence is copied and thus expanded into a larger number of polynucleotide molecules, e.g., by reverse transcription, polymerase chain reaction, and ligase chain reaction.

The terms “bacteriophage” and “phage” are used interchangeably herein and refer to viruses which infect bacteria. By the use of the terms “bacteriophage library” or “phage library” as used herein, is meant a population of bacterial viruses comprising heterologous DNA, i.e., DNA which is not naturally encoded by the bacterial virus.

As used herein, the term “bait protein” means any desired protein to which contact with a phage display library is desired.

As used herein, the term “encoding” refers to the inherent property of specific sequences of nucleotides in a polynucleotide, such as a gene, a CDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, the term “interacting protein” is meant any protein which binds to a bait protein.

As used herein, a “polynucleotide” means a single or parallel and anti-parallel strands of a nucleic acid. Thus, a polynucleotide may be either single-stranded or a double-stranded nucleic acid.

As used herein, the term “promoter sequence” means a nucleic acid sequence which is required for expression of a gene product operably linked to the promoter sequence. In some instances, this sequence may be the core promoter sequence and in other instances, this sequence may also include an enhancer sequence and other regulatory elements which are required for expression of the gene product. The promoter sequence may, for example, be one which expresses the gene product in a tissue specific manner.

By describing two polynucleotides as “operably linked” is meant that a single-stranded or double stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

### Description

The invention should not be construed to be limited to any particular bait protein. It is a property of virtually all known proteins that they interact with each other. Thus, the bait protein may be any protein which is useful for the identification of other proteins with which it interacts.

In one embodiment, the bait protein comprises a detectable label, which in a preferred embodiment, comprises biotinylated lysine. Biotinylated lysine is detected with horseradish peroxidase-conjugated streptavidin and an ECL detection kit.

The detectable label is added to the bait protein during in vitro translation of MRNA encoding the bait protein and may comprise any detectable label which may be added to a protein during the process of in vitro translation. The invention is not limited to any particular type of detectable label, but rather includes detectable label that can be attached to a protein.

In a second embodiment, the bait protein is part of a fusion construct wherein the gene encoding the fusion protein is fused to a sequence encoding a binding protein, which in a preferred embodiment, comprises glutathione S transferase.

The interacting protein is present on the surface of a phage, which phage comprises DNA encoding the same. Preferably, the DNA encoding the interacting protein is cDNA, but, the invention is not limited to cDNA, and includes any and all DNAs which encode protein that can be expressed on the surface of a phage.

Although the examples provided herein presents data obtained using a phage display library comprising T7 bacteriophage, the invention is not limited to the use of this bacteriophage alone. Rather, the invention includes bacteriophage which are capable of displaying a protein expressed therefrom on their surface, wherein the protein is then available for binding to a bait protein.

The assay of the invention can be automated. For example, through the use of a 96 well plate, multiple samples of bait proteins and the phage display library may be mixed at one time. Washing and detection and isolation of bound phage may be accomplished using automated technology available in the art.

Once the interacting protein has been identified and DNA encoding this protein has been obtained, it is possible to mutate the DNA, express the protein therefrom and select proteins which exhibit high affinity for the bait protein. Thus, the invention also includes a method of accelerated protein evolution. Proteins which are generated using this method are useful for diagnostic and therapeutic purposes.

The invention is further described in detail by reference to the following experimental examples. These examples are provided for purposes of illustration only, and are not intended to be limiting unless otherwise specified. Thus, the invention is not limited to the following examples, but rather, includes any and all variations which are evident as a result of the teaching provided herein.

### EXAMPLE 1

### Identification of Protein via in Vitro Translation System and Phage Display Library

We have used an in vitro translated protein to rapidly identify and clone interacting proteins from a cDNA library expressed on the surface of bacteriophage T7. Transportin (TRN1), the nuclear transport receptor for M9-containing proteins including the major hnRNP proteins A1 and A2, was produced in vitro in a coupled transcription-translation system using rabbit reticulocyte lysate supplemented with biotinylated lysine-tRNA. An aliquot of the translation mixture was then incubated with an aliquot of the phage display library and the biotin-TRN1, together with the phages that specifically interact with it, was retrieved from the mixture with streptavidin-conjugated beads. The selected phage were amplified and the cycle was repeated several times resulting in a vast enrichment of phage bearing TRN1-interacting proteins, mostly A1 and A2. This method makes it possible to construct in a short period of time a network of protein interactions on a large scale without the need to first express the proteins of interest in bacteria and purify them. It also makes it possible to study the effect of additional components (proteins, RNA, DNA, ligands, etc.) and to vary the experimental conditions, and facilitates the process of accelerated evolution of proteins in vitro.

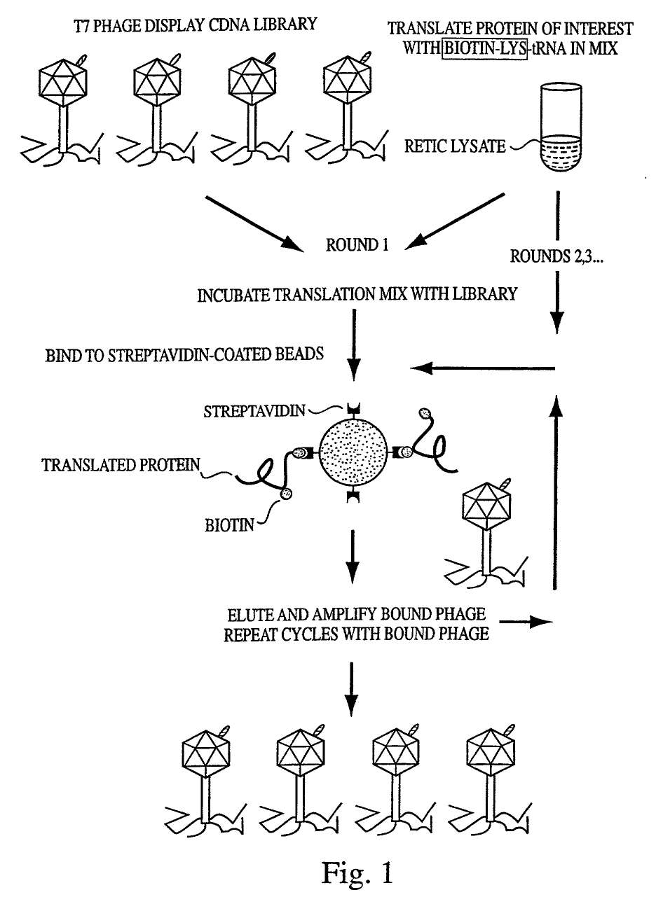

To circumvent the difficulties encountered using prior art methods of identifying interactive proteins, and to facilitate rapid construction of networks of interactions of proteins on a large scale, we developed a method that will make it possible to rapidly identify interactions without the need to first express and purify the protein of interest as a recombinant fusion protein in bacteria or another heterologous system. The approach we devised (illustrated in FIG. 1) is based on the use of a protein translated in vitro, produced from a cDNA transcribed in vitro (or a portion of such a protein) as a bait to isolate an interacting protein from a cDNA (or genomic) library expressed on the surface of a phage display library. The in vitro translated protein bait is produced with a tag so that it can be readily retrieved from the translation mix. An aliquot of the phage library is incubated with the translation mix containing the bait under the desired conditions, and the phages that bind to the bait protein are isolated and amplified. This cycle can be repeated several times to enrich the mixture for phages bearing the interacting proteins. Because the phage contains the DNA sequence that encodes the protein that is expressed on its surface, this procedure results in the rapid cloning of the interacting proteins.

More specifically, for phage selection experiments, an aliquot of the library was thawed and refreshed by inoculating 10 microliters of phage (4×10⁸ pfu) into 1 milliliter of E. coli BLT5403 culture at O.D.₆₀₀=0.1 in LB/Amp. The culture was incubated at 37° C. with shaking until complete lysis occurred (1 hour). The lysate was clarified by centrifugation in an Eppendorf tabletop centrifuge for 2 minutes and placed on ice until use. Biotinylated proteins were produced in a coupled in vitro transcription translation system (Promega TNT) containing 25 microliters rabbit reticulocyte lysate, 2 microliters TNT buffer, 1 microliters T7 RNA polymerase, 1 microliter RNasin RNase inhibitor, 1 microliter biotin-RNA-lysine (10 mM)(Boehringher Mannheim #1559478), 1 microliter Amino acid mix without lysine (50 mM)(Boehringher Mannheim #1559478), 1 microliter DNA plasmid (1 μg/μl), and 17 microliters distilled H₂O to a final volume of 50 microliters. Incubation was at 30° C. for 1.5 hours.

A 10 microliter aliquot of the refreshed phage library lysate and 25 microliters of the biotinylated protein product were added to a 1.5 milliliter Eppendorf tube containing 500 microliters of 1×Transport Buffer (TB, 20 mM HEPES pH 7.3, 110 mM KOAc, 5 mM NaOAc, 2 mM MgOAc, 0.5 mM EGTA, 2 mM DTT and 1 mg/ml each aprotinin, pepstatin and leupeptin); and incubated with gentle mixing for 15 minutes at 4° C. A 40 microliter suspension of streptavidin beads (Boehring Mannheim #1529188), prepared by washing twice with 1 milliliters of 1×PBS and blocked with 3% BSA in 1×PBS by mixing in an end-to-end rotator at room temperature for 0.5 hour, and further rinsed twice with 1×TB twice), was added, and incubation was continued with gentle mixing for 10 minutes at room temperature. The beads were collected by centrifugation and the supernatant was discarded. The beads were washed 5 times, each with 1 milliliter of 1×TB. After the last wash, the buffer was removed using a standard pipette tip, and the liquid was carefully drained with a fine long gel loading tip to minimize loss of beads. Bound phage were eluted by treatment with 30 microliters of 4M urea for 10 minutes at room temperature with occasional vortexing after which the beads were briefly spun and the supernatant was removed and used to inoculate a 1 milliliter of BUT5403 culture at O.D.=0.1. To amplify the selected phage, incubation was carried out at 37° C. with shaking until complete lysis occurred (approximately 1 to 1½ hour). For storage, 20 milliliters of chloroform were added and the lysate was placed at 4° C. Repeat cycles were carried out as described.

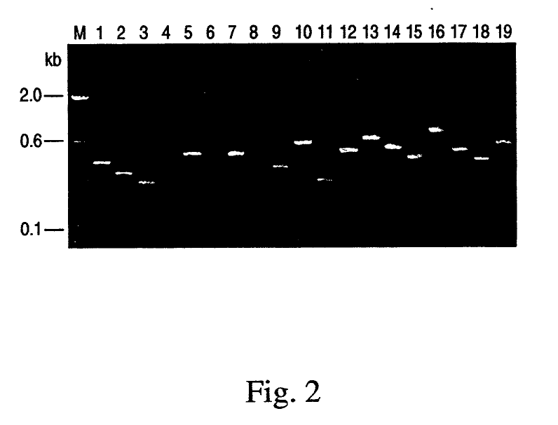

In the example described here, we produced the translated bait protein in the presence of biotin-lysine-charged tRNA so that the resulting protein contains biotin and can thus be readily isolated with streptavidin immobilized on Sepharose beads, or on 96-well plates. The cDNA library was constructed from HeLa mRNA and expressed as fusions with the coat protein on the surface of bacteriophage T7. Total poly(A) MRNA was prepared from HeLa using the Straight A′Soligo(dT) magnetic beads system (Novagen, Madison, Wis.), and cDNA was produced by directional cloning using random priming, and a cDNA T7 phage library was constructed in the T7 cloning vector (T7Select1-1, Novagen, Madison, Wis.). The T7 phage is particularly suited for this purpose as it has a short lytic cycle and is highly resistant to conditions that may be needed for high stringency washes and for elution of the selected phage from the bait (Studier, 1972, Science 176:367-376; Kruger and Schroeder, 1981, C. Microbiol. Rev. 45:9-51). FIG. 2 shows an ethidium bromide stained agarose gel on which PCR-amplified inserts of 18 randomly selected phage were separated by electrophoresis. The directional, random-primed library contained 2×10⁶ independent clones, and greater than 95% of the phages contained inserts, ranging in size from 0.2-1.2 kb.

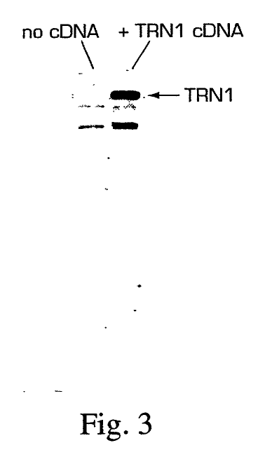

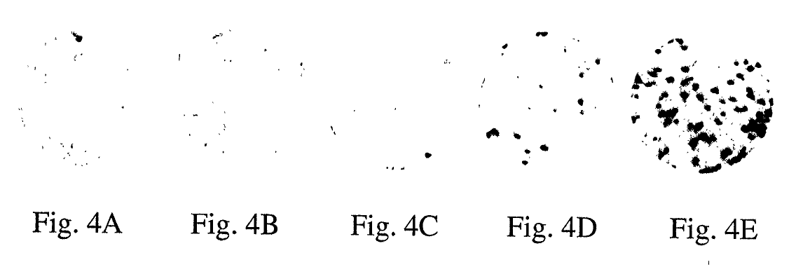

As a bait for a test case, we used transportin, the nuclear import receptor for the hnRNP proteins A1 and A2 (Pinol-Roma et al., 1988, Genes Dev. 2:215-227; Dreyfuss et al., 1993, Annu. Rev. Biochem. 62:289-321) Transportin (TRN1) is a 97 kD protein that specifically binds to the 38 amino acid M9 nuclear transport sequence present in A1 and A2 (Siomi and Dreyfuss, 1995, J. Cell Biol. 129:551-560; Michael et al., 1995, Cell, 83:415-422). For reference, we used recombinant glutathione-S-transferase (GST)-TRN1 fusion produced in E. coli. A blot showing the translated product, biotin-TRN1, detected with streptavidin-horseradish peroxidase is shown in FIG. 3. Note that there are endogenous biotinylated proteins in the reticulocyte lysate, but they do not interfere with the selection of biotin-TRN binding proteins (see below). Binding and washing were carried out for 15 minutes at room temperature in transport buffer as TRN1 functions well in nuclear import of A1 and A2 under these conditions (Pollard et al., 1996, Cell 86:985-994). Because M9-containing fragments of A1 and A2 were expected to be major interactors of TRN1, we used plaque hybridization with full length A1 and A2 cDNAs (Burd, 1989, Proc. Natl. Acad. Sci. USA 86:9788-9792) to monitor for enrichment of these sequences after each selection cycle. The results of filter hybridization of aliquots of selected phage plated after each round are shown in FIG. 4. Both GST-TRN1 and in vitro translated biotin-TRN1, showed very significant enrichment after two rounds of selection and the majority of the plaques were hybridization positive after three rounds. Importantly, in vitro translated biotin-TRN1 was as efficient as GST-TRN1.

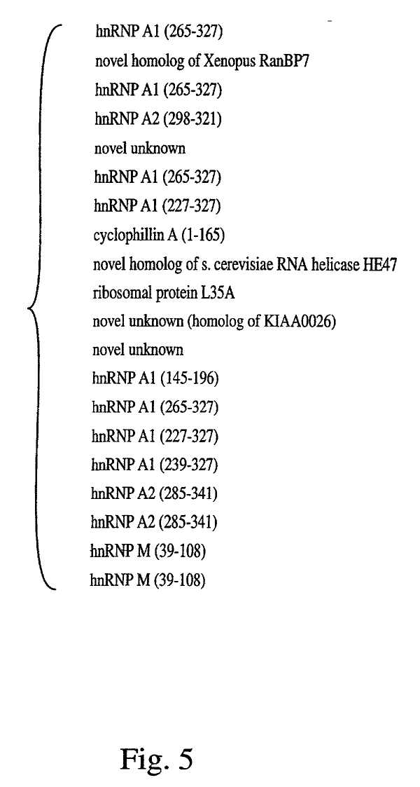

To confirm the identity of the biotin-TRN1-selected phage, we PCRamplified the inserts of 20 randomly picked phage after the fourth round of selection and determined their sequences. The sequence information, presented in FIG. 5, demonstrates that over 75% of the proteins selected with biotin-TRN1 are M9-containing proteins, including A1 and A2. Direct binding experiments will be required to determine if the other selected clones are bonafide TRN1-interacting proteins or simply non-specific background binders.

### EXAMPLE 2

### Identification of Proteins via Fusion Protein and Phage Display Library

A fusion protein was used to rapidly identify and clone interacting proteins from a cDNA library expressed on the surface of bacteriophage T7. The fusion construct comprised a CDNA encoding bacteriophage T7 polymerase promoter sequence for in vitro transcription, fused upstream of a sequence encoding glutathione S transferase (GST), which is a high affinity microbial glutathione binding protein, to generate a fusion construct designated GST-TRN1. The GST sequence was further fused in frame to TRN1. The mRNA was produced by transcription in vitro, in a reticulocyte lysate coupled in vitro transcription-translation system.

An aliquot of the mixture was incubated with an aliquot of a phage display library. GST-TRN1 and phages that specifically interact with it were retrieved from the mixture using glutathione-Sepharose beads.

The cDNA library was constructed from HeLa mRNA and expressed as fusions with the coat protein on the surface of bacteriophage T7. Six rounds of phage selection amplification were performed and ten isolated plaques obtained following the last round of selection were sequenced.

For phage selection experiments, an aliquot of the library was thawed and refreshed by inoculating 10 microliters of phage (4×10⁸ pfu) into 1 milliliter of E. coli BLT5403 culture at O.D.₆₀₀ =0.1 in LB/Amp. The culture was incubated at 37° C. with shaking until complete lysis occurred (1 hour). The lysate was clarified by centrifugation in an Eppendorf tabletop centrifuge for 2 minutes and placed on ice until use. A 10 microliter aliquot of the refreshed phage library lysate and 25 microliters of the fusion protein product were added to a 1.5 milliliter Eppendorf tube containing 500 microliters of 1×Transport Buffer (TB, 20 mM HEPES pH 7.3, 110 mM KOAc, 5 mM NaOAc, 2 mM MgOAc, 0.5 mM EGTA, 2 mM DTT and 1 mg/ml each aprotinin, pepstatin and leupeptin); and incubated with gentle mixing for 15 minutes. at 4° C. A 40 microliter suspension of glutathione-Sepharose beads, (prepared by washing twice with 1 milliliter of 1×PBS and blocked with 3% BSA in 1×PBS by mixing in an end-to-end rotator at room temperature for 0.5 hour, and further rinsed twice with 1×T twice), was added, and incubation was continued with gentle mixing for 10 minutes at room temperature. The beads were collected by centrifugation and the supernatant was discarded. The beads were washed 5 times, each with 1 milliliter of 1×TB. After the last wash, the buffer was removed using a standard pipette tip and the liquid was carefully drained with a fine long gel loading tip to minimize loss of beads. Bound phage were eluted by treatment with 30 microliters of m. urea for 10 minutes at room temperature with occasional vortexing after which the beads were briefly spun and the supernatant was removed and used to inoculate a 1 milliliter of BUT5403 culture at O.D.=0.1. To amplify the selected phage, incubation was carried out at 37° C. with shaking until complete lysis occurred (approximately 1 to 1½ hr). For storage, 20 milliliters of chloroform were added and the lysate was placed at 4° C. Repeat cycles were carried out as described above.

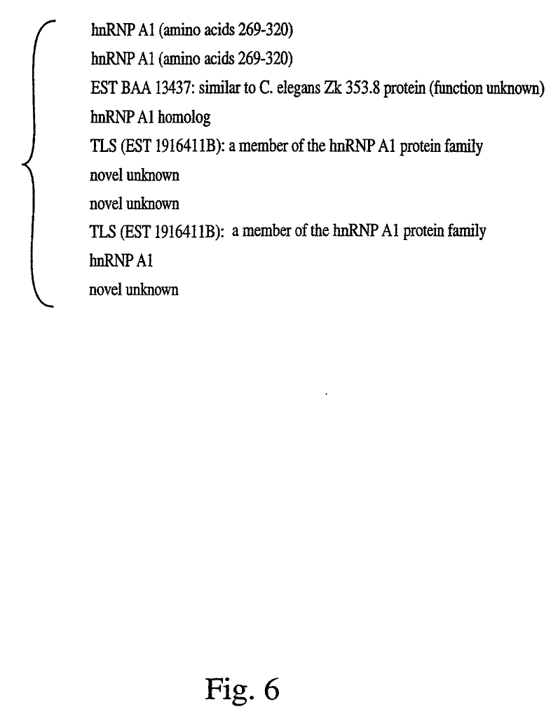

As indicated in FIG. 6, the majority of the identified clones were either A1 itself, specifically including the TRN 1 binding M9 sequence, or related proteins, such as TLS, known to contain an M9-like domain, and are thus expected to bind to TRN1.

The experiments described above, as well as selection of interacting proteins using other baits, establish a simple and powerful system for identification and cloning of interacting proteins. The optimal conditions for selection may vary for different proteins and will have to be determined experimentally. This system makes it possible to manipulate the experimental environment by adding proteins, RNA, DNA and ligands, as well as by adjusting the specific conditions, such as time, temperature, and ionic strength. This method provides quick results, in that they may be obtained in less than two days. The method readily lends itself to scaling up and automation. For example, it is possible to use a large array of baits immobilized on 96 well plates, and employ robotics to carry out the phage binding and washing steps, and add bacteria directly to the wells for phage amplification in situ. Starting with cDNAs generated with promoters suitable for transcription in vitro in a coupled transcription-translation system, it is possible to construct a map of a whole network of interactions for a large number of proteins in a relatively short period of time. This will be helpful for analysis of novel proteins about which no functional information is available, as is increasingly the case with rapidly accumulating genomic and EST sequence information. The analysis of the proteins protein's interactors may provide important clues as to its likely function. As an alternative to biotin-lysine it is possible to use a variety of tags with which to retrieve the translated proteins, by simply transcribing them from DNA constructs that contain the desired tag as a fusion with the bait and use specific high affinity reagents (e.g. antibodies) to the tag.

Finally, using mutagenesis on the selected phage as a starting point, a new sub-library can be prepared for accelerated molecular evolution of proteins. By selecting winners out of such a library after multiple rounds of selection so that competition between the selected phage is established, it is possible to obtain useful high affinity reagents for the original protein bait.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of isolating an interacting protein, said method comprising
contacting a bait protein with a phage display library, wherein said phage display library comprises candidate interacting proteins expressed on the surface of phage in said library,
separating phage in said phage display library which bind said bait protein from phage in said phage display library which do not bind said bait protein, and
isolating said interacting protein from said phage which bind said bait protein.

2. The method of claim 1, wherein said bait protein is obtained by in vitro translation of RNA encoding said bait protein.

3. The method of claim 2, wherein said bait protein has a detectable label attached thereto.

4. The method of claim 3, wherein said detectable label is biotinylated lysine.

5. The method of claim 1, wherein said phage display library comprises bacteriophage T7.

6. The method of claim 1, wherein said bait protein is encoded in a gene fused to a cDNA encoding a binding protein.

7. The method of claim 6, wherein said binding protein is glutathione S transferase.

8. A method of obtaining an interacting protein capable of binding to a bait protein with high affinity, said method comprising
contacting a bait protein with a phage display library, wherein said phage display library comprises candidate interacting proteins expressed on the surface of phage in said library,
separating phage in said phage display library which bind said bait protein from phage in said phage display library which do not bind said bait protein, isolating DNA encoding said interacting protein from said phage which bind said ait protein, mutating said DNA to generate an interacting protein encoded thereby which is capable of binding to said bait protein with high affinity.

9. The method of claim 8, wherein said bait protein is obtained by in vitro translation of RNA encoding said bait protein.

10. The method of claim 9, wherein said bait protein has a detectable label attached thereto.

11. The method of claim 10, wherein said detectable label is biotinylated lysine.

12. The method of claim 8, wherein said phage display library comprises bacteriophage T7.

13. The method of claim 8, wherein said bait protein is encoded in a gene fused to a cDNA encoding a binding protein.

14. The method of claim 13, wherein said binding protein is glutathione S transferase.
